(19) United States
(12) Patent Application Publication
(10) Pub. No.: US 2021/0052493 A1
(43) Pub. Date: Feb. 25, 2021

(54) NON-AQUEOUS ANTI-PARASITIC OINTMENT FOR OPHTHALMOLOGICAL USE

(71) Applicant: Joseph Sinclair, Scott Depot, WV (US)

(72) Inventor: Joseph Sinclair, Scott Depot, WV (US)

(21) Appl. No.: 16/999,276

(22) Filed: Aug. 21, 2020

Related U.S. Application Data
(60) Provisional application No. 62/889,618, filed on Aug. 21, 2019.

(51) Int. Cl. A61K 9/06; A61K 31/7048; A61K 47/44

(52) U.S. Cl. CPC A61K 9/06; A61K 9/0048; A61K 47/44; A61K 31/7048

(57) ABSTRACT

A nonaqueous ointment for the treatment of persons who are identified by their ophthalmologist to have an infection of common parasites in and around the follicles of the eyelash. The invention is a highly viscous mixture of inert components and antiparasitic powder, as well as a method of application considering the unique composition's qualities. The unique ointment and application method eradicate the pathogens entirely by taking into account their general behavior and lifecycle. Once an infection is identified using instruments readily available to eye doctors in their offices, the ointment is applied once daily to the base of the eye lashes by an ointment tube with an ophthalmic tip. The ointment is applied shortly before bed, over the course of several months. Existing methods of treatment have additional side effects and are largely ineffective, with the parasites often recovering in number after treatment has ceased. The current invention far exceeds these levels of therapeutic effectiveness and offers a permanent solution to the infection of the follicles.

### CROSS-REFERENCE TO OTHER APPLICATIONS

This application does not claim the benefit of any other application.

### BACKGROUND OF THE INVENTION

Most people have some variety of parasite or mite living on their skin. When the immune system becomes aware of these parasites, an inflammatory response and discomfort typically follow and subsist until the parasite is eliminated. While these infections are caused by organisms which are invisible to the naked eye, during examinations ophthalmologists have a unique opportunity to observe the affected area under significant magnification and can identify the infections easily. The treatment of these infections is often difficult due to the sensitive nature of the eye and the hardy nature of the parasites. There is no existing approved topological method of treating infections, such as Demodex folliculorum mites or similar parasitic pathogens, that live in and around the eye lashes and eyebrows. Patients and doctors are often so plagued by the problem that other topical prescription medicines are explored for use off label, even when counter indicated for ophthalmological use. Existing systemic treatments are generally inadequate and often fail to solve the underlying issue. The current invention remedies this problem by using a novel compound to serve as a topical ointment applied in a unique fashion upon the sensitive areas, alleviating the malady when prescribed by an ophthalmologist and applied to the sensitive areas, removing the need to seek further treatment entirely.

### BRIEF SUMMARY OF THE INVENTION

Clinical treatment of Ocular Demodex folliculorum has proven difficult. Demodex mites that live in and around the eye lash follicles are inherently difficult to kill for numerous reasons. They stay mostly embedded in the follicle while light is present, meaning they spend most of their time deeply rooted in the follicle hiding from danger. Furthermore, they subside on dead skin, which makes systemic treatments that are introduced through the blood by and large ineffective. As a result, existing treatments often fail to fully eradicate the mites and they eventually multiply back to their original number. Once the immune system is aware of the mites, the only way to curb the response besides rendering the entire immune system ineffective is to kill off the pathogen entirely. Given the difficulty presented in doing so, many patients today live in a great deal of discomfort due to demodicosis of the eye lash.

The invention consists of a unique composition of an antiparasitic powder with petrolatum and mineral oil to create a non-viscous ointment that is safe for use around the eye and can be easily prescribed by an ophthalmologist. The targeted infections are caused by microscopic pathogens that would not be visible to a general practitioner, but can be readily identified under magnification typically employed in eye exams or other ocular medical settings. Existing suspensions of antiparasitic powders, such as but not limited to ivermectin, are water based and are not approved for ophthalmologic use. Water based ointments and creams, while more readily available for absorption by the skin, may also irritate the eye. Furthermore, they are applied in a fashion that is unlikely to kill the parasites entirely or effectively. The current novel composition, applied in a unique fashion, has clinical benefits that strongly outweigh the competition and have the potential to eradicate the underlying infection.

The primary ingredients of the invention are powdered antiparasitic compounds and a non-aqueous oil-based medium. In one possible manifestation of the invention, the compound ivermectin comprises a small portion of the mixture by mass. The remainder consists of a thick oil-based medium composed of roughly equal parts white petrolatum and mineral oil, as well as small amounts of a drying agent such as lanolin alcohol. The mixture is then placed in a tube with an ophthalmic tip and administered to the base of the eyelashes by the patient. While antiparasitic compounds like ivermectin are generally only neurotoxic to the parasites that they kill and not to humans, they can irritate sensitive tissues, so avoiding direct contact with the eye remains critical.

While many of these microscopic parasites are somewhat common and live on the body without incident, they can affect the areas around the eyelash follicles at a disproportionate rate that rises to the level of an infection. These infections often cause discomfort in the eyelid and can be spotted by small build ups of parasitic wastes around the base of the eyelash, followed by swelling, pain, and other immune responses which result in a loss of function due to discomfort. Patients plagued by this issue have repeatedly failed to find relief using existing methods. By providing a safe to use topical treatment specific to the eyelashes, the current invention grants eye doctors the ability to rectify the problem in a single consultation with a single prescription. Once patients apply the compound as directed, the present invention does not provide a simple treatment but rather a permanent cure and total relief from symptoms.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise embodiments shown in the drawings.

FIG. 1 details one process by which the invention can be made from known ingredients. The ingredients, listed in the black box at the top of the figure, are combined and prepared for application according to steps 1 through 10. This sort of instruction sheet is the guide which would be employed by a compound pharmacist to enable them to create the combined pharmaceutical product currently described within this application.

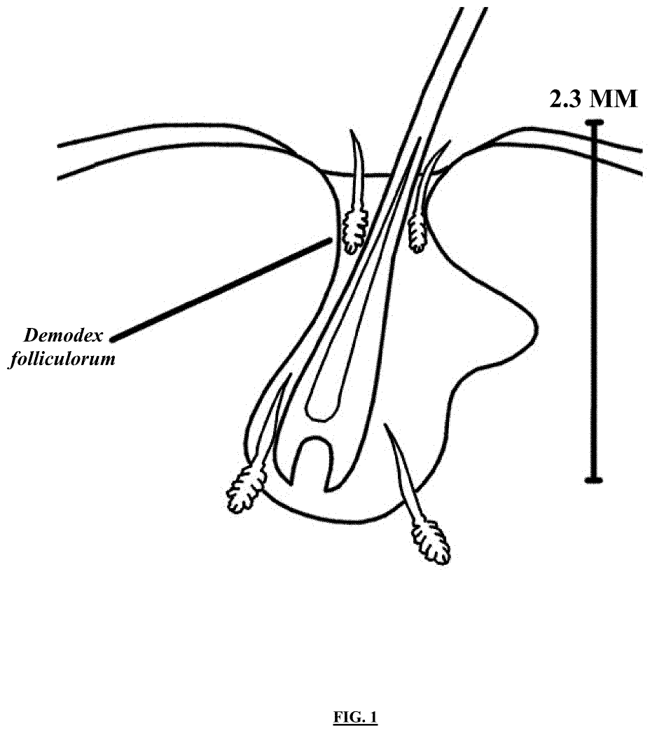

FIG. 2 shows a scale drawing of a parasitic infection of the eye lash follicle by the skin mite Demodex folliculorum which would be treatable with the current invention. The base of the follicle is approximately 2.3 millimeters beneath the surface of the skin. Demodex follicullorum, which reach a size of between 0.3 and 0.4 millimeters, are shown in numerous places around the follicle which they might occupy at different times. Demodex feeds on dead skin cells, so the mite needs to temporarily leave the safety of the follicle during the course of its life. Demodex enter and leave the follicle at its base, next to the hair. When observed under light and magnification, the tails of severable mites are typically visible as they flee the skin's surface in search of darkness. Mating occurs at the follicle opening, while ovum are deposited within the follicle itself. The mite's entire life cycle occurs between the base of the follicle and the surface of the skin.

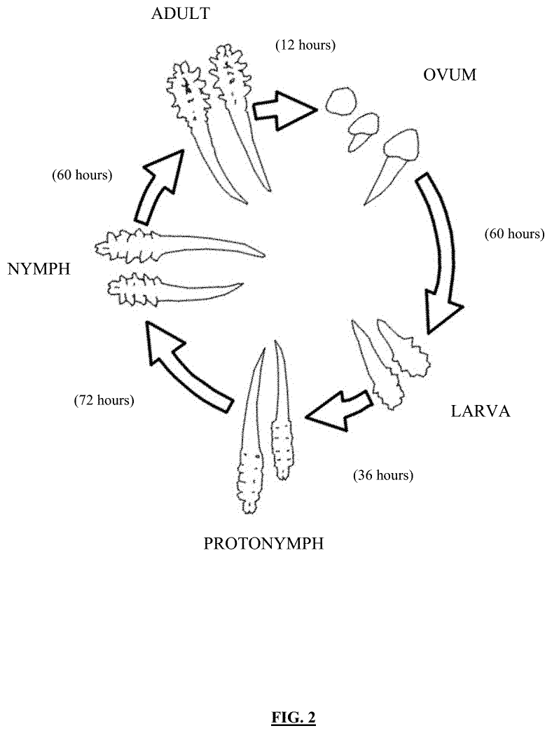

FIG. 3 details the life cycle of a typical Demodex mite. Estimates of the species' lifecycle range between one week and a month, with most experts hypothesizing a timeframe of roughly ten (10) days. Life starts for a mite of this variety when a female lays a clutch of 15 to 20 eggs inside the hair follicle, near the sebaceous gland. During the ovum and larval stages, the organism resides inside the eyelash follicle. After a few days, the still developing Demodex protonymphs begin to become mobile, as they will need to feed in order to grow and complete their life cycle. After a few days, the mites begin to resemble their adult form as they grow through their nymph stage of development and eventually become sexually mature adults. Adulthood is brief for a mite, characterized solely by fulfilling its living purpose and mating near the surface of the eyelash. Mites then die, either around the base of the eye lash or within the follicle. Their bodies and exoskeletons decompose, giving rise to deposits visible by the naked eye at the base of a patient's eyelashes as contributing to the inflammatory immune response. A critical advantage of the present invention is that, due to its unique nature and application, it has the potential to completely eradicate the mite population on the host when applied over the full time of the life cycle by killing the parasites before they can reproduce.

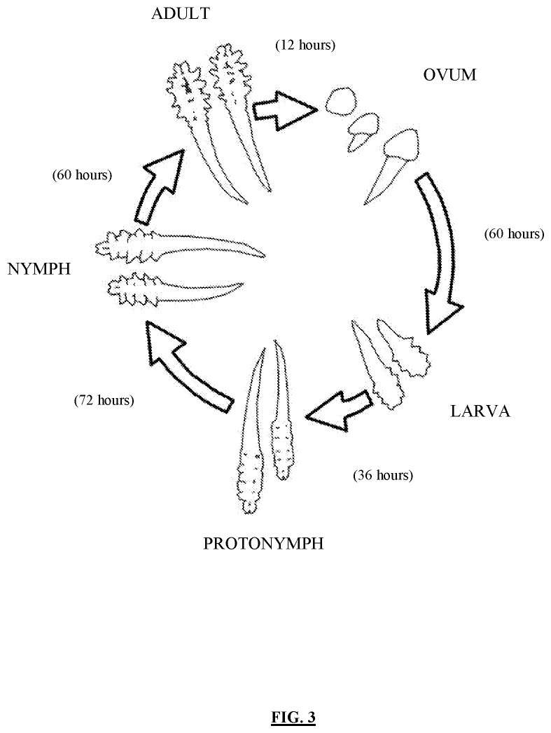

### DETAILED DESCRIPTION

Infections of the eyelashes are embarrassing and painful. More often than not, they are caused by Demodex folliculorum or a similar microscopic parasite embedded in the follicle of the hair that feed on dead skin. These small animals undergo their entire lifecycle in and around the eyelash in a matter of weeks, leaving decomposing dead mites in and around the hair follicle. In addition to these build ups, such infections can cause swelling and discomfort as a result of the body's immunological response. Current methods of treatment range considerably, with the most basic approach being to simply scrub the area repeatedly with gentle soaps to exfoliate and starve the parasites while avoiding the use of greasy make-ups that might worsen the infection. In most circumstances, this is insufficient to give any type of real relief and doctors or patients attempt to use increasingly aggressive approaches such as systemic treatment such as used to treat mange in animals, or off label use of topological creams that could damage ocular tissues. Even these treatments fail in the long run, as temporary relief under these methodologies often gives way to recurrent infections.

The present invention encompasses a topical means of treating infections of the eyelash, or other sensitive areas surrounding the eye, consisting of a common antiparasitic powder suspended in a mixture of nonaqueous oils. In one possible version of the invention, the antiparasitic powder consists of ivermectin, and the nonaqueous mixture of oils consists of a combination of white petrolatum and mineral oil. This iteration may further include a drying agent, such as powdered lanolin alcohol, in order to prevent the mixture from becoming aqueous and increasing in viscosity. The resultant product is a unique low viscosity topical ointment for delicate application of antiparasitic compounds around the eye. Once applied to the base of the eyelashes prior to going to bed, the ointment settles around the base of the lash as the patient sleeps.

The novel mixture is prepared by first heating and melting a portion of the nonaqueous oil medium in order to increase viscosity. Once the mixture is heated, the powdered components of the mixture are added and mixed. The remainder of the oil medium may be added at this stage, and additional heating and mixing steps may be employed to create a homogenous mixture before the resulting ointment is subsequently allowed to cool to room temperature. If the powder does not fully dissolve, it may be necessary to run the cooled ointment through a clean ointment mill. It is imperative that the compound remain nonaqueous, so prior to using a mill or any other equipment, said piece of equipment should be cleaned with alcohol and dried thoroughly. Once the powder has been fully dissolved, the ointment is placed in a metal ointment tube which is fixed with an ophthalmological tip.

Ivermectin and other compounds within the class of antiparasitic treatments called antihelmintics achieve their desired therapeutic effect by attacking the nervous systems of organisms such as mites, roundworms, flatworms, tapeworms, flukes, and several other parasites known to the scientific community as helminths. While they are selectively neurotoxic, thus they are not deadly to the mammalian hosts they are used to treat, they can irritate sensitive tissues and membranes. The only other alternative is to administer the compound systemically, however such a course of treatment greatly expands the patient's natural biology to exposure to the compound and can give rise to a host of negative side effects. By allowing for a means to apply the compound locally the current invention presents a significant improvement over these methods.

Helminths and mites are thought to be responsible for numerous skin and hair follicle ailments. Virtually all human bodies are home to some species of these parasites, and the species are generally unique to and exist solely within the human body as well. Antiparasitic compounds are employed to treat conditions that are either known to be or thought to be caused by these parasites. These compounds have been administered at the systemic level orally for some time in the veterinary industry and, more recently, in humans. Where topical treatments have been used for some ailments, such as in the treatment of rosacea, they are applied by way of aqueous creams to allow for them to be spread and absorbed through the skin directly. To date, no topical ointment of this fashion has been postulated or created for the treatment of follicular infections near the eye. The unique nature of these infections requires a unique course of treatment.

When an ophthalmologist identifies and diagnoses a parasitic infection in the skin or hair follicles nearest to the eye, they can prescribe the invented ointment to be applied by the patient, once prepared or purchased from a pharmacy. When applied, typically one time a day prior to bed, unlike existing antiparasitic treatments, the ointment attaches to and sits on the surface of the skin or follicle. Most mites, such as demodex, are mostly active at night as they draw back into hair follicles when exposed to light. They conduct most of the activity that encompasses their short lives during this time. Medications such as hydrochloric acid that are applied during the day often fail to kill the parasites as they are hidden safely away during application, however as the patient sleeps and the pathogens become active, they come into contact with the antiparasitic compounds and ultimately die. Skin parasites have complete lifecycles that range from a couple weeks to a month or two, meaning that a course of treatment with this invention which lasts for such a length of time has the potential to eliminate the infection entirely.

The unique nature of this nonaqueous ointment allows for total eradication of the parasitic population on the eyelashes. Most parasites reproduce over the course of several weeks, meaning that if even one generation can survive treatment, eventually the whole population recovers, and the infection persists. The present invention accounts for this issue by being applied over a period of time and in a fashion that is designed to eliminate the population in a far more effective fashion than existing treatment methods. By using a thick ointment applied over the base of the eyelashes just prior to bedtime over a course of two months, Demodex or similar parasites are killed during the window of time in which they leave the hair follicle under the cover of darkness. While even applying in this fashion is not fool proof, as it may only account for the small portion of the parasite's population that is currently in the vulnerable segment of its lifecycle, applying over a period of time greater than a full generation kills a vast majority of the individuals and effectively cures the infection. What results is a noninvasive and final cure to a typically recurrent affliction which also minimizes side-effects common amongst the other less effective treatment methods which are currently available.

What is claimed is:

1. A composition used for the treatment of ocular parasitic infections in the form of an ointment comprising:
an anti-parasitic compound; and
a viscous non-aqueous medium.

2. The composition of claim 1 where the ocular parasitic infection treated by the ointment is caused by demodex mites.

3. The composition of claim 1 further comprising a drying agent.

4. The composition of claim 3 where the drying agent is lanolin alcohol powder.

5. The composition of claim 1 where the anti-parasitic compound is ivermectin.

6. The composition of claim 1 where the non-aqueous medium is a mixture of mineral oil and white petrolatum.

7. A method of preparing an ointment for the treatment of ocular parasitic infections, with the method comprising the steps of:
preparing a non-aqueous medium;
adding an effective amount of an antiparasitic compound;
mixing the contents of the ointment; and
placing the ointment in an ointment tube with an ophthalmic tip.

8. The method of claim 7 where the ocular parasitic infection treated by the ointment is caused by demodex mites.

9. The method of claim 7 where the method further comprises the step of adding a drying agent before missing the contents and placing the ointment in an ointment tube with an ophthalmic tip.

10. The method of claim 9 where the drying agent is lanolin alcohol powder.

11. The method of claim 7 where the anti-parasitic compound is ivermectin.

12. The method of claim 7 where the non-aqueous medium is a mixture of mineral oil and white petrolatum.

13. A composition for use as an antiparasitic ointment for ocular infections comprising:
an anti-parasitic compound; and
a viscous non-aqueous medium.

14. The composition for use as an antiparasitic ointment to treat ocular infections of claim 13 where the ocular infection treated by the ointment is caused by demodex mites.

15. The composition for use as an antiparasitic ointment to treat ocular infections of claim 13 further comprising a drying agent.

16. The composition for use as an antiparasitic ointment to treat ocular infections of claim 15 where the drying agent is lanolin alcohol powder.

17. The composition for use as an antiparasitic ointment to treat ocular infections of claim 13 where the anti-parasitic compound is ivermectin.

18. The composition for use as an antiparasitic ointment to treat ocular infections of claim 13 where the non-aqueous medium is a mixture of mineral oil and white petrolatum.
